(19) United States
(12) Patent Application Publication
(10) Pub. No.: US 2021/0077055 A1
(43) Pub. Date: Mar. 18, 2021

(54) METHOD FOR CONTROLLING RADIOGRAPHIC IMAGING SYSTEM

(71) Applicant: BIOSENSTECH INC., Daejeon (KR)

(72) Inventor: SEUNG IK JUN, Gyeonggi-do (KR)

(21) Appl. No.: 16/572,992

(22) Filed: Sep. 17, 2019

(51) Int. Cl. A61B 6/00

(52) U.S. Cl. CPC A61B 6/548; A61B 6/56

(57) ABSTRACT

A method for controlling a radiographic imaging system, in which the radiographic imaging system includes a radiation source, a radiation detector, and a mobile device, and in which the method for controlling the radiographic imaging system is performed at the radiation detector, the method includes (a) monitoring a preset radiation exposure danger area to determine whether a living organism is detected within the radiation exposure danger area, and (b1) when the living organism is not detected, sending information that radiographic imaging is ready to the mobile device and sending a command to operate the radiation source to the radiation source. The method may prevent radiation exposure of an operator or a third party in advance by automatically controlling an operation of a radiographic imaging system.

### TECHNICAL FIELD

The present disclosure relates to a method for controlling a radiographic imaging system, and more particularly, to a method for controlling a radiographic imaging system for preventing radiation exposure of an operator or a third party during radiographic imaging.

### BACKGROUND

A radiographic imaging system is a device that photographs the inside of a subject by using a property in which radiation such as X-rays are absorbed or transmitted depending on the characteristics of materials. The radiographic imaging system receives radiation that has penetrated the subject or occurred inside the subject, and then, generates a radiographic image based on an electrical signal output according to the received radiation to provide an operator with the image information inside the subject.

The radiographic imaging system is used in various industrial fields because the structure of the subject may be easily grasped. For example, the radiographic imaging system is used to detect lesions inside a human body in a hospital or the like. In addition, the radiographic imaging system is used to check inside baggage at an airport checkpoint. The radiographic imaging system is also used for non-destructive testing of objects and components installed in factories or outdoors.

In the non-destructive test conducted at the factory or outdoors, an operator is always in the danger of radiation exposure. In order to prevent the danger of radiation overexposure of such an operator, Korean Patent Publication No. 10-0529181 discloses a technique for a device to prevent radiation overexposure. However, the technique described in Korean Patent No. 10-0529181 only solves the danger of the operator ignoring (or not knowing) the radiation overexposure alarm through the intervention of a colleague or a manager. It does not prevent the danger of radiation exposure in advance or solve the danger of radiation exposure due to the entry of a third party who is not an operator to the workplace.

### SUMMARY

The present disclosure has been made to solve the above-described problems, and to provide a technique for automatically controlling an operation of a radiographic imaging system to prevent radiation exposure of an operator or a third party.

In addition, the present disclosure provides a technique for the operator to easily grasp the control result of the radiographic imaging system.

Further, the present disclosure is to provide a technique that the operator may easily perform an action according to the control result of the radiographic imaging system.

Other objects of the present disclosure will become more apparent through the preferred embodiments described below.

According to an aspect of the present disclosure, a method for controlling a radiographic imaging system, in which the radiographic imaging system includes a radiation source, a radiation detector, and a mobile device, and in which the method is performed at the radiation detector, the method including: (a) monitoring a preset radiation exposure danger area to determine whether a living organism is detected within the radiation exposure danger area; and (b1) when the living organism is not detected, sending information that radiographic imaging is ready to the mobile device and sending a command to operate the radiation source to the radiation source.

Here, the command to operate the radiation source may be sent when a signal for sending the command to operate the radiation source may be received from the mobile device.

Here, the step (b1) may include sending information that the radiographic imaging has started to the mobile device when radiation emitted from the radiation source is detected.

Here, (b2) when the living organism is detected, the method for controlling the radiographic imaging system may further include sending information that the living organism was detected to the mobile device.

According to further aspect of the present disclosure, a method for controlling a radiographic imaging system, in which the radiographic imaging system comprises a radiation source, a radiation detector, and a mobile device, and in which the method is performed at the radiation detector, the method including: (a) sending a command to operate the radiation source to the radiation source; (b) monitoring a preset radiation exposure danger area to determine whether a living organism is detected within the radiation exposure danger area; and (c) when the living organism is detected, sending a command to stop an operation of the radiation source to the radiation source, and sending information that the living organism was detected to the mobile device.

Here, the step (c) may include sending a command to re-operate the radiation source to the radiation source when a command to resume radiographic imaging is received from the mobile device.

Here, after sending the information that the living organism was detected, the step (c) may include sending a command to re-operate the radiation source and sending information that radiographic imaging resumed to the mobile device when no living organism is detected within the radiation exposure danger area by monitoring the danger area again.

Here, after sending the information that the living organism was detected, the step (c) may further include sending information that the radiographic imaging is ready to resume to the mobile device when no living organism is detected within the radiation exposure danger area by monitoring the danger area again.

According to another aspect of the present disclosure, a recording medium recorded a method for controlling a radiographic imaging system, in which the radiographic imaging system includes a radiation source, a radiation detector, and a mobile device, and in which the method is performed at the radiation detector, the recording medium recording and providing a computer program for the method for controlling the radiographic imaging system, in which the computer program includes functions of (a) monitoring a preset radiation exposure danger area to determine whether a living organism is detected within the radiation exposure danger area; and (b1) when the living organism is not detected, sending information that radiographic imaging is ready to the mobile device and sending a command to operate the radiation source to the radiation source.

According to another aspect of the present disclosure, a recording medium recorded a method for controlling a radiographic imaging system, in which the radiographic imaging system includes a radiation source, a radiation detector, and a mobile device, and in which the method is performed at the radiation detector, the recording medium recording and providing a computer program for the method for controlling the radiographic imaging system, in which the computer program includes functions of (a) sending a command to operate the radiation source to the radiation source; (b) monitoring a preset radiation exposure danger area to determine whether a living organism is detected within the radiation exposure danger area; and (c) when the living organism is detected, sending a command to stop an operation of the radiation source to the radiation source, and sending information that the living organism was detected to the mobile device.

The present disclosure has the benefit of preventing radiation exposure of an operator or a third party in advance by automatically controlling an operation of a radiographic imaging system.

In addition, the present disclosure has the benefit that the operator may easily grasp the control result of the radiographic imaging system.

Further, the present disclosure has the benefit that the operator may easily perform an action according to the control result of the radiographic imaging system.

### BRIEF DESCRIPTION OF THE DRAWINGS

FIG. 1 is a view showing the configuration of a radiographic imaging system according to an embodiment of the present disclosure.

FIGS. 2 to 6 are flowcharts showing a method for controlling a radiographic imaging system according to a first to fifth embodiments of the present disclosure.

### DETAILED DESCRIPTION

The present disclosure may be applied with various modifications and may have various embodiments. Accordingly, the present disclosure is intended to be illustrated by specific embodiments in the drawings and described in detail in the detailed description. However, it should be understood that the present disclosure is not intended to be limited to particular embodiments, but includes all modifications, equivalents, and alternatives falling within the spirit and scope of the present disclosure. In the following description, when it is considered that the detailed description for the related known art may blur the gist of the present disclosure, the detailed description thereof will be omitted.

The terms first, second, etc. may be used to describe various elements, but the elements should not be limited by the terms. The terms are used only for the purpose of distinguishing one component from another.

The term used herein is only used to describe the particular embodiments, and is not intended to limit the present disclosure. Unless the context clearly means otherwise, the singular expression includes plural expression. It should be understood that, herein, the terms “include” or “have,” etc. are intended to specify that there is a stated feature, number, step, operation, component, part, or a combination thereof herein, and it does not exclude in advance the possibility of presence or addition of one or more other features, numbers, steps, operations, components, parts, or combinations thereof. Hereinafter, embodiments of the present disclosure will be described in detail with reference to the accompanying drawings.

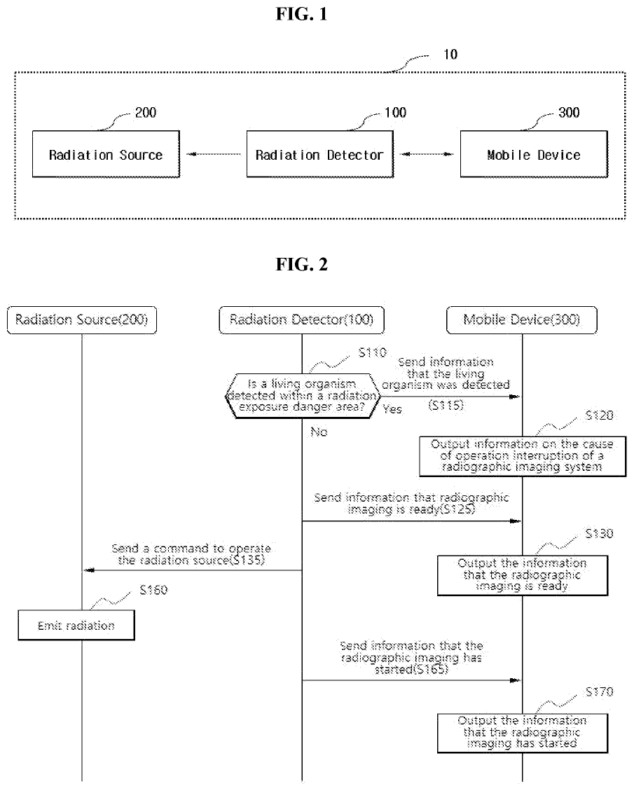

FIG. 1 is a view showing the configuration of a radiographic imaging system according to an embodiment of the present disclosure.

As shown in FIG. 1, a radiographic imaging system 10 includes a radiation detector 100, a radiation source 200, and a mobile device 300. The radiation detector 100, the radiation source 200, and the mobile device 300 may be connected through a local area network to share information with each other. Here, the local area network may be applied without limitation as long as the radiation detector 100, the radiation source 200, and the mobile device 300 share information with each other, such as Wi-Fi, direct Wi-Fi, Bluetooth, or RF.

The radiation source 200 is a device that emits radiation for non-destructive testing. Here, the non-destructive testing is a test method that checks the internal cracks or defects of a subject without destroying the subject using radiation. In the non-destructive test, the radiation source 200 emits radiation toward the detector 100, and the subject is positioned between the radiation source 200 and the detector 100. The radiation may be in various forms depending on the type of test and the type of test subject. For example, the radiation may be X-rays, neutron rays, alpha rays, beta rays, gamma rays, and the like.

The radiation detector 100 receives radiation transmitted the subject and converts the radiation into an electrical signal, and the converted electrical signal becomes a digital image. The radiation detector 100 according to the present disclosure includes a module for monitoring a living organism. Here, the living organism is usually humans such as operators, third parties around the workplace, but may also be animals. The module for monitoring the living organism detects living organisms around the radiographic imaging workplace. In particular, the module for monitoring the living organism monitors the radiation exposure danger area predetermined according to the direction of radiation emission, the amount of radiation energy, and the environment of the workplace, and detects the presence of the living organism within the radiation exposure danger area. The module for monitoring the living organism may be an ultrasonic sensor, an infrared sensor, a camera module, etc. to detect the living organism, and any other device capable of detecting the living organism may be applied without limitation. A plurality of modules for monitoring the living organism may be used to designate areas of exposure danger in various forms.

The mobile device 300 receives information delivered from the radiation detector 100 and outputs the received information to a display provided in the mobile device 300 so that the operator may view the received information. In addition, the mobile device 300 receives a command from the operator and delivers it to the radiation detector 100. The mobile device 300 may be a smart band, a smartphone, a smart pad, a portable laptop, or the like, and as long as a device is portable for the operator and has a communication function for exchanging information with the radiation detector 100, it may be applied to the mobile device 300 without limitation.

Hereinafter, a method of preventing radiation exposure of an operator or a third party through control of the radiographic imaging system according to the present disclosure will be described with reference to FIGS. 2 to 6.

FIG. 2 is a flowchart showing a method for controlling a radiographic imaging system according to a first embodiment of the present disclosure.

As shown in FIG. 2, when pairing between devices is completed so that the radiation detector 100, the radiation source 200, and the mobile device 300 may share information, the operator drives the radiographic imaging system 10, the radiation detector 100 determines whether a living organism is detected in a radiation exposure danger area (S110). As described above, the radiation exposure danger area may be set in various ways depending on the emission direction of the radiation, the magnitude of the radiation energy, and the environment around the workplace.

As a result of determination, when a living organism is detected in the radiation exposure danger area, the radiation detector 100 sends information on the detection of the living organism to the mobile device 300 (S115). Here, the mobile device 300 outputs information on a cause of the operation stop of the radiographic imaging system so that the operator may know that the living organism is detected in the radiation exposure danger area and thus the radiographic imaging system may not operate (S120). For example, the mobile device 300 may output a message “The radiographic imaging has been stopped because a person is present in the radiation exposure danger area.” on the display. Reasons for stopping the operation of the radiographic imaging system 10 may be various, such as failure of the device or communication failure. In this embodiment, it is intended to prevent radiation exposure of the operator or the third party in advance by monitoring the radiation exposure danger area.

When no living organism is detected within the radiation exposure hazard area in step S110, the radiation detector 100 sends information indicating that the preparation of radiographic imaging is completed to the mobile device 300 (S125). Here, the mobile device 300 outputs information on the completion of radiographic imaging preparation that the operator may know that the living organism is not detected in the radiation exposure danger area and thus the radiographic imaging is ready (S130). For example, the mobile device 300 may output a message “The radiographic imaging starts soon because no person is present as a result of monitoring the radiation exposure danger area” on the display.

After step S125 or at the same time, the radiation detector 100 sends a command to emit radiation to the radiation source 200 (S135). In response to the command, the radiation source 200 emits radiation toward the subject (S160). When the radiation detector 100 detects radiation transmitted through the subject, the radiation detector 100 sends information indicating that the radiographic imaging has started to the mobile device 300 (S165). Here, the mobile device 300 outputs information indicating that the radiographic imaging has started so that the operator may know that the radiographic imaging has started (S170). For example, the mobile device 300 may output a massage “Radiographic imaging has started. Do not enter the work area until the radiographic imaging is completed.”

Although not shown in FIG. 2, the radiographic imaging is performed for a preset time after step S170, and when the radiographic imaging is completed, the mobile device 300 may output information indicating that the radiographic imaging is completed. Regarding to the completion of the radiographic imaging, the imaging may be recognized as being completed when a preset time for radiographic imaging elapsed. Further, regarding to the completion of the radiographic imaging, the imaging may be recognized as being completed when the preset time for radiographic imaging elapsed, the radiation detector 100 sent a command to stop the emission of radiation to the radiation source 200, and then no radiation emitted from the radiation source 200 is detected.

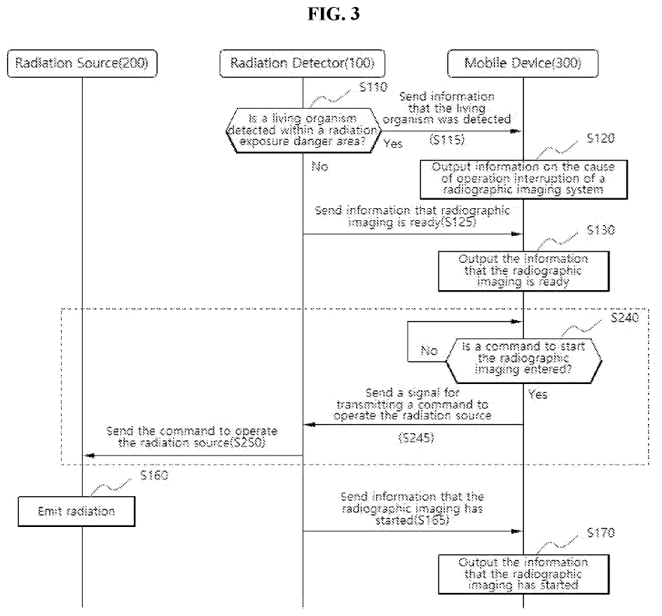

FIG. 3 is a flowchart showing a method for controlling a radiographic imaging system according to a second embodiment of the present disclosure.

As shown in FIG. 3, the same steps as the first embodiment described with reference to FIG. 2 are performed in the second embodiment of the present disclosure, other than steps S240 to S250 in which dashed boxes are shown. Therefore, hereinafter, redundant descriptions will be omitted or briefly mentioned.

In step S130, a mobile device 300 outputs information indicating that the preparation of radiographic imaging is completed on a display. Thereafter, the mobile device 300 waits until a command to start radiographic imaging is input from an operator. Here, the mobile device 300 may provide a selection menu through which the operator may input the command to start the radiographic imaging. For example, it may output a selection message “Yes” or “No” together with “Ready for radiographic imaging. Do you want to start radiographic imaging?” to wait for the operator to select.

When the command to start the radiographic imaging is input from the operator in step S240, the mobile device 300 sends a signal to the radiation detector 100 to transmit a command to operate a radiation source (S245). After that, the radiation detector 100 sends the command to operate the radiation source to the radiation source 200 (S250). In response to the command, the radiation source 200 emits radiation toward the subject (S160). When the radiation detector 100 detects radiation transmitted through the subject, the radiation detector 100 sends information indicating that the radiographic imaging has started to the mobile device 300 (S165). Here, the mobile device 300 outputs information indicating that the radiographic imaging has started so that the operator may know that the radiographic imaging has started (S170).

In the second embodiment of the present disclosure, unlike the first embodiment, after the radiation detector 100 monitors the radiation exposure danger area, the operator looks at the radiation exposure danger area with the naked eye once more, and then, the radiographic imaging may be performed. Therefore, it has a benefit that the radiographic imaging may be made safer.

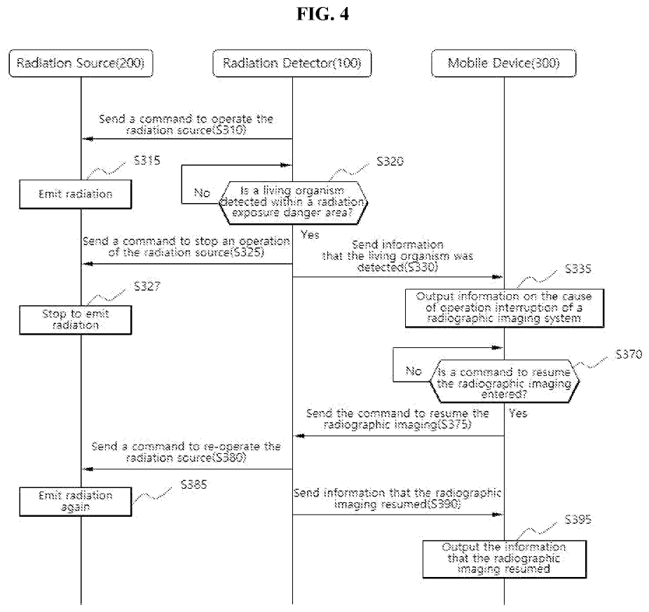

FIG. 4 is a flowchart showing a method for controlling a radiographic imaging system according to a third embodiment of the present disclosure.

When the operator drives the radiographic imaging system 10, the radiation detector 100 sends the command to operate the radiation source to the radiation source 200 in step S310, and in response to the command, the radiation source 200 emits radiation toward the subject (S315). Further, the radiation detector 100 determines whether the living organism is detected in the radiation exposure danger area immediately after step S310 in a continuous or preset period (S320).

When the living organism is detected within the radiation exposure area as a result of the determination, the radiation detector 100 sends a command to stop the operation of the radiation source to the radiation source 200 (S325), and the radiation source 200 stops emitting radiation in accordance with the command (S327). Here, when a signal of the command to operate the radiation source is a signal that continues continuously (or continuously with any period), and when no signal of the command to operate the radiation source has been received continuously, the radiation source 200 may recognize this as the command to stop the operation of the radiation source. Alternatively, the signal of the command to operate the radiation source and the signal of the command to stop the operation of the radiation source may be another type of signal. In other words, when the signal of the command to stop the operation of the radiation source, which has a different form from the signal of the command to operate the radiation source, is received, the radiation source 200 may recognize it as a command to finish the operation of the radiation source.

Further, when the living organism is detected in the radiation exposure danger area, the radiation detector 100 sends information on the detection of the living organism to the mobile device 300 (S330). Here, the mobile device 300 outputs information on the cause of the operation stop of the radiographic imaging system so that the operator may know that the living organism is detected in the radiation exposure danger area (S120). For example, the mobile device 300 may output a message “Detection of people in the radiation exposure danger area!!! Check the safety of your radiographic imaging workplace.” on the display. Here, the operator may check the safety state of the radiographic workplace according to the message and then resume the radiographic imaging if there is no abnormality in safety.

After step S335, the mobile device 300 waits until the command to start the radiographic imaging is input from the operator. Here, the mobile device 300 may provide the selection menu through which the operator may input the command to resume the radiographic imaging. For example, the mobile device 300 may output the selection message “Yes” or “No” together with “Did you check the safety of your workplace? Do you want to resume the radiographic imaging?” to wait for the operator to select.

When the command to resume the radiographic imaging is input from the operator in step S370, the mobile device 300 sends the command to resume the radiographic imaging to the radiation detector 100 (S375). After that, the radiation detector 100 sends a command to re-operate the radiation source to the radiation source 200 (S380). In response to the command, the radiation source 200 emits radiation again toward the subject (S385). When the radiation detector 100 detects radiation transmitted through the subject, the radiation detector 100 sends information indicating that the radiographic imaging has resumed to the mobile device 300 (S390). Here, the mobile device 300 outputs information indicating that the radiographic imaging has resumed so that the operator may know that the radiographic imaging has resumed (S395).

According to the third embodiment of the present disclosure, the radiation detector 100 monitors the radiation exposure danger area. When an abnormal situation is occurred, first, the radiographic imaging is stopped, and the operator looks at the radiation exposure danger area with the naked eye once more, and then resumes the radiographic imaging. Therefore, it has a benefit that the radiographic imaging may be made safer, the operator looks at the radiation exposure danger area with the naked eye once more, and then, the radiographic imaging may be performed. Therefore, it has a benefit that the radiographic imaging may be made safer.

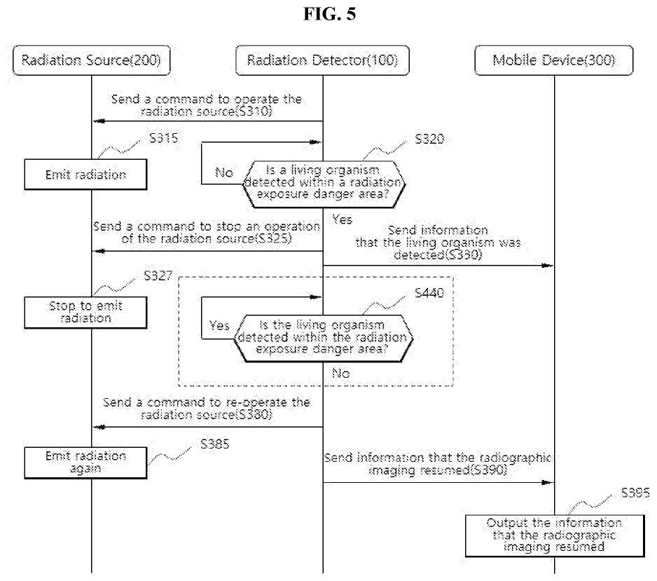

FIG. 5 is a flowchart showing a method for controlling a radiographic imaging system according to a fourth embodiment of the present disclosure.

Referring to FIG. 5, in the fourth embodiment of the present disclosure, steps other than S440 indicated by a dotted line box are described with reference to FIG. 4, and thus, redundant descriptions will be omitted or briefly mentioned below.

When the living organism is detected within the radiation exposure danger area in step S320, the radiation detector 100 sends the command to stop the operation of the radiation source to the radiation source 200 (S325), and thus, the radiographic imaging is stopped. Thereafter, the radiation detector 100 determines whether the living organism is detected in the radiation exposure danger area, continuously or repeatedly at a preset period (S440). When the living organism is not detected within the radiation exposure area as a result of the determination, the radiation detector 100 sends the command to re-operate the radiation source to the radiation source 200 (S380). In response to the command, the radiation source 200 emits radiation again toward the subject (S385). When the radiation detector 100 detects radiation transmitted through the subject, the radiation detector 100 sends information indicating that the radiographic imaging has resumed to the mobile device 300 (S390). Here, the mobile device 300 outputs information indicating that the radiographic imaging has resumed so that the operator may know that the radiographic imaging has resumed (S395).

According to the fourth embodiment of the present disclosure, when the radiation detector 100 monitors the radiation exposure danger area and the living organism is detected, the radiographic imaging is first stopped. Thereafter, the radiation detector 100 monitors until the living organism disappears in the radiation exposure danger area. When monitoring reveals that the living organism has disappeared in the radiation hazardous area, the radiographic imaging resumes. Unlike the third embodiment, the fourth embodiment of the present disclosure is advantageous in that the imaging, stop, and resumption of the radiographic imaging are automatically made without the involvement of an operator.

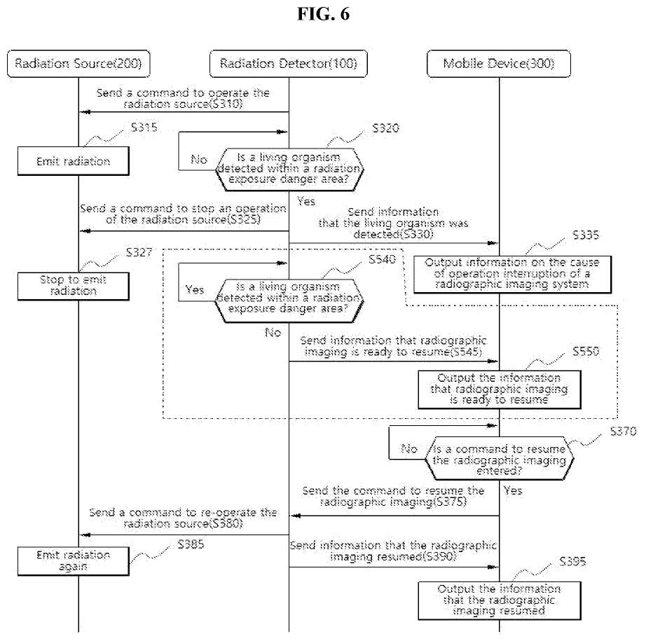

FIG. 6 is a flowchart showing a method for controlling a radiographic imaging system according to a fifth embodiment of the present disclosure.

Referring to FIG. 6, in the fifth embodiment of the present disclosure, steps other than S540 to S 550 indicated by a dotted line box are described with reference to FIG. 4, and thus, redundant descriptions will be omitted or briefly mentioned below.

When the living organism is detected within the radiation exposure danger area in step S320, the radiation detector 100 sends the command to stop the operation of the radiation source to the radiation source 200 (S325), and thus, the radiographic imaging is stopped. Thereafter, the radiation detector 100 determines whether the living organism is detected in the radiation exposure danger area, continuously or repeatedly at a preset period (S540). If it is determined that no living organism is detected within the radiation exposure area, the radiation detector 100 sends information indicating that the radiographic imaging is ready to resume to the mobile device 300 (S545). Here, the mobile device 300 outputs information indicating that the radiographic imaging is ready to resume so that the operator may know that no more living organism is detected in the radiation exposure danger region and thus the radiographic imaging may be resumed (S550). For example, the mobile device 300 may output a message “A person is no longer be detected in the radiation exposure danger area, and thus, the radiographic imaging may resume.” Here, the operator may check the safety state of the radiographic imaging workplace according to the message and then resume the radiographic imaging if there is no abnormality in safety.

After step S550, the mobile device 300 waits until the command to resume the radiographic imaging is input from the operator. Here, the mobile device 300 may provide the selection menu through which the operator may input the command to resume the radiographic imaging. When the command to resume the radiographic imaging is input from the operator in step S370, the mobile device 300 sends the command to resume the radiographic imaging to the radiation detector 100 (S375). After that, the radiation detector 100 sends a command to re-operate the radiation source to the radiation source 200 (S380). In response to the command, the radiation source 200 emits radiation again toward the subject (S385). When the radiation detector 100 detects radiation transmitted through the subject, the radiation detector 100 sends information indicating that the radiographic imaging has resumed to the mobile device 300 (S390). Here, the mobile device 300 outputs information indicating that the radiographic imaging has resumed so that the operator may know that the radiographic imaging has resumed (S395).

The method for controlling the radiographic imaging system according to the present disclosure described above may be embodied as computer readable codes on a computer readable recording medium. The computer readable recording medium includes all kinds of recording devices in which data that may be read by a computer system is stored. Examples of the computer-readable recording medium include ROM, RAM, CD-ROM, magnetic tape, floppy disk, optical data storage, and the like, and those implemented in the form of carrier waves such as transmission over the Internet. The computer readable recording medium may also be distributed over network coupled computer systems so that the computer readable code is stored and executed in a distributed fashion.

The foregoing preferred embodiments of the present disclosure have been disclosed for the purpose of illustration, and it would be possible for those skilled in the art to make various modifications, alterations and additions thereof within the spirit and scope of the invention. Further, it would be appreciated that such modifications, alterations and additions fall within the scope of the following claims.

What is claimed is:

1. A method for controlling a radiographic imaging system comprising a radiation source, a radiation detector, and a mobile device, the method comprising:
(a) monitoring, with the radiation detector, a preset radiation exposure danger area to determine whether a living organism is detected within the radiation exposure danger area; and
(b1) when the living organism is not detected, sending information that radiographic imaging is ready to the mobile device and sending a command to operate the radiation source to the radiation source.

2. The method of claim 1, wherein, the command to operate the radiation source is sent from the radiation detector to the radiation source when a signal for sending the command to operate the radiation source is received, by the radiation detector, from the mobile device.

3. The method of claim 1, wherein the step (b1) comprises sending information that the radiographic imaging has started to the mobile device when radiation emitted from the radiation source is detected at the radiation detector.

4. The method of claim 1, further comprising:
(b2) when the living organism is detected, sending information that the living organism was detected to the mobile device.

5. A method for controlling a radiographic imaging system comprising a radiation source, a radiation detector, and a mobile device, the method comprising:
(a) sending a command to operate the radiation source to the radiation source;
(b) monitoring, with the radiation detector, a preset radiation exposure danger area to determine whether a living organism is detected within the radiation exposure danger area; and
(c) when the living organism is detected, sending a command to stop an operation of the radiation source to the radiation source, and sending information that the living organism was detected to the mobile device.

6. The method of claim 5, wherein the step (c) comprises sending a command to re-operate the radiation source to the radiation source when a command to resume radiographic imaging is received from the mobile device.

7. The method of claim 5, further comprising, after sending the information that the living organism was detected, sending a command to re-operate the radiation source and sending information that radiographic imaging resumed to the mobile device when no living organism is detected within the radiation exposure danger area by monitoring the danger area again.

8. The method of claim 6, further comprising, after sending the information that the living organism was detected, sending information that the radiographic imaging is ready to resume to the mobile device when no living organism is detected within the radiation exposure danger area by monitoring the danger area again.

9. A computer readable medium recorded with a computer program for executing in a computer system a method for controlling a radiographic imaging system that comprises a radiation source, a radiation detector, and a mobile device, the method comprises:
(a) monitoring, with the radiation detector, a preset radiation exposure danger area to determine whether a living organism is detected within the radiation exposure danger area; and
(b1) when the living organism is not detected, sending information that radiographic imaging is ready to the mobile device and sending a command to operate the radiation source to the radiation source.
